(19) United States
(12) Patent Application Publication
(10) Pub. No.: US 2014/0038884 A1
(43) Pub. Date: Feb. 6, 2014

(54) ANTIVIRAL PEPTIDES

(71) Applicants: Tiziana Cabras, Monserrato (IT); Claudio Casoli, Parma (IT); Massimo Castagnola, Roma (IT); Rosanna Inzitari, Roma (IT); Renato Longhi, Milano (IT); Irene Messana, Monserrato (IT); Paola Ronzi, Milano (IT); Alberto Vitali, Roma (IT)

(72) Inventors: Tiziana Cabras, Monserrato (IT); Claudio Casoli, Parma (IT); Massimo Castagnola, Roma (IT); Rosanna Inzitari, Roma (IT); Renato Longhi, Milano (IT); Irene Messana, Monserrato (IT); Paola Ronzi, Milano (IT); Alberto Vitali, Roma (IT)

(73) Assignees: UNIVERSITA 'DEGLI STUDI DI CAGLIARI, Cagliari (IT); UNVERSITA' CATTOLICA DEL SACRO CUORE, Milano (IT); CONSIGLIO NAZIONALE DELLE RICERCHE, Roma (IT); UNVERSITA' DEGLI STUDI DI MILANO, Milano (IT)

(21) Appl. No.: 13/981,940

(22) PCT Filed: Jan. 30, 2012
(86) PCT No.: PCT/IB12/50415

§ 371 Date: Oct. 15, 2013

(30) Foreign Application Priority Data
Jan. 28, 2011 (IT) RM2011A000035

(51) Int. Cl. C07K 14/435

(52) U.S. Cl. CPC C07K 14/435
USPC 514/3.8; 530/326; 536/23.5

(57) ABSTRACT

The present invention relates to selected proline-rich peptides of salivary derivation with a strong antiviral activity and also an anti-reservoir activity with respect to the HIV virus, said peptides as medicaments for the treatment, prevention and eradication of HIV on human beings, pharmaceutical compositions comprising at least one of said peptides, pharmaceutical kits comprising at least one of said peptides and therapeutic methods for the treatment, prevention and eradication of HIV on human beings.

The present invention relates to selected proline-rich peptides of salivary derivation with a strong antiviral activity and also an anti-reservoir activity with respect to the HIV virus accountable for Acquired Immune deficiency Syndrome (AIDS), said peptides as medicaments for the treatment, prevention and eradication of HIV on human beings, pharmaceutical compositions comprising at least one of said peptides, pharmaceutical kits comprising at least one of said peptides and therapeutic methods for the treatment, prevention and eradication of HIV on human beings.

### PRIOR ART

In the last years, several biologically active proline-rich peptides (PRPs, proline-rich proteins) have been isolated from insects, amphibians and mammals. These peptides are characterized by a >50% proline content and constitute the first barrier of defense of the innate immune system. Their main activity is of antimicrobial nature, in spite of the fact that some of them proved multifunctional, supplementing their antimicrobial activity with the ability to modulate the formation of protein complexes and to intervene in various cell functions, among which oxidative stress, cytoskeleton formation and cell motility. Antimicrobial proline-rich peptides have originally been isolated from insects, such as drosocins, isolated from Hymenoptera, Lepidoptera and Hemiptera and Diptera, which kill above all Gram-negative bacteria without causing membrane lysis. The sequence of these peptides is characterized by repetition of the Pro-Arg-Pro tripeptide. Another typical member of the family are pyrrhocoricines, isolated from Pyrrhocoris apterus, and apidaecins, isolated from Apis mellifera. These peptides exhibit a broad degree of conservation of the PRP-PHPRI/L sequence in the C-terminus end. Proline-rich peptides have also been isolated from mammals. Among those, cathelicidins and bactenecines should be mentioned. The best-known member of cathelicidins is peptide PR39, first isolated from pig intestine, and of which analogs have subsequently been isolated from spleen neutrophiles (Shi J. et al 1994; Bonetto V. et al 1999). PR39 is of remarkable interest, as in addition to antibacterial activity it modulates various cell processes (oxidative stress, cytoskeleton formation). Bactenecins can also be classified in the same family of cathelicidins. Two peptides, Bac 5 and Bac 7, belong to the bactenecine group. These peptides, isolated from bovine neutrophils, are respectively 42-e 59-residues long and 75% homologous to PR39. They exert antimicrobial activity against Gram-negative bacteria, but can also act as antiviral agents against Herpes simplex (Zerman A. et al 1987). Proline-rich peptides can be involved in the interaction with profilin-like protein recognition domains SH3, WW, EVH1, GYF, which play a key role in various biological processes. In mammals' saliva, proline-rich proteins (PRPs), both acidic and basic ones, represent a relevant class. PRPs represent almost 60% by weight of the proteins present in human parotid secretion, basic PRPs are a complex class of peptides generated by pre-secretory processes of pro-protein fragmentation. (Messana I. et al 2004). Moreover, after secretion, these peptides encounter a further fragmentation process by endogenous and exogenous oral proteases (oral flora). (Messana I. et al 2008; Helmerhorst E J et al 2008; Vitorino R et al 2009).

To date, the exact role of basic PRPs has not been fully understood, though some defense activities towards Candida fungal infections have been reported (O'Sullivan J M et al 1997).

Robinovitch M R et al 2001 and Stangler T et al 2007 describe the role of some proline-rich peptides, from human parotid saliva and of synthetic nature, as potential HIV inhibitors. In the first paper it is reported that some chromatographic fractions containing human basic PRP preparations possess antiviral activity against strains HIV-1 Bal (laboratory strain with monocyte-macrophage tropism, a characteristic of isolates in primary infections) and LAI (laboratory strain with T-lymphotropic tropism, strains usually isolated in patients with full-blown AIDS). In particular, it is partially described a protein exhibiting such activity. Of the protein are reported a relatively high molecular weight of about 37 kDa and the ability to bind glycoprotein gp120 (a glycoprotein for recognition by human lymphocyte receptors), in case this be used in the purification process by affinity chromatography. Robinovitch M R et al 2001 reports no information about the protein structure, apart from a high content in proline residues.

In the second paper an artificial proline-rich peptide is described, obtained by phage library technique, inside which there is a particular sequence, reported in the paper, with the ability to inhibit bonding between the Nef protein of the HIV virus and the SH3 domain of the protein kinase Hck of hematopietic cells, and therefore to prevent the onset of infection. In the paper it is hypotesized the general possibility of utilizing proline-rich peptides as potential novel therapeutic agents.

U.S. Pat. No. 5,981,720 describes the inhibitory activity of two salivary basic glycoproteins, denominated CON-1 and CON-2, and of some of their fragments on the alpha-glucosidase enzyme of HIV-1. In particular, the U.S. patent ascribes this inhibition to a GGNK subfragment (SEQ ID NO 4), of the above-indicated basic proteins and attributes the inhibition of HIV-1 infection to such activity. According to said patent, the ability to inhibit glucosidase activity causes non-glycosilation of viral envelope proteins, with the entailed impossibility for the virus to penetrate the host cell (paragraph 9, line 42). In short, the patent hypothesizes the use of peptides comprising the above-reported tetra-amino acid sequence, with alpha-glucosidase inhibition activity, as antiretroviral drugs.

Today, therapy for HIV infection and AIDS mainly makes use of the combination of three classes of antiviral drugs directed against structural proteins of the virus which do not have a counterpart in human cells: nucleoside (AZT, abacavir, stavudine) and non-nucleoside (nevirapina, efavirenz) reverse-transcriptase inhibitors, integrase inhibitors (raltegravir) and viral protease inhibitors (ritonavir, atazanavir, darunavir, nelfinavir). Recently, a new class of drugs inhibiting the virus-host cell fusion process (maraviroc, enfuvirtide) was introduced.

Today, the most widely practiced therapeutic approach is that known as HAART (Highly Active Antiretroviral Therapy), based on co-administration of three drugs belonging to different classes. This type of combined therapy is able to reduce, below the sensitivity of the detection method, plasma viremia levels in most of the patients treated for three years. However, HAART drawbacks are represented on the one side by the very high cost, and on the other side by logistic difficulties imposed by a very complicated dosage regimen, as well as, to some patients, by the high toxicity. Last but not least, the emergency dictated by the appearance or resistant HIV strains should be mentioned, representing the main cause of therapeutic failures. In this regard, it has recently been reported that more than 70% of treated individuals carry HIV variants that are resistant to one or more retroviral drugs.

Moreover, another limitation of the drugs currently in use is the inability to eliminate latent viral reservoirs, and therefore to completely eradicate the virus from the organism. In fact, the HIV virus makes use of a replication mechanism which makes a total eradication thereof extremely difficult. In fact, it is now known that the virus integrates in some organs of the human body in which it proves inaccessible even to the action of drugs which effectively inhibit key aspects of the viral life cycle, thereby generating viral reservoirs which, above all when generated in the early stages of the infection, are one of the major obstacles in the treatment of the infection.

A detailed study on HIV viral reservoirs and on the current state of the art is related in the report by Richard Jeffreys on the International AIDS Society's Workshop of 2010, enclosed herein in its entirety. The report shows how in the current state of things a strategy for controlling HIV reservoirs, and therefore for effectively treating HIV infections, is not available yet.

As also described in Nitin K Saksena et al. 2010, even though the treatment of HIV-infected persons with highly active antiretroviral therapy (HAART) leads to a control of plasma viremia to levels below the detectable threshold (i.e., <40 copies/plasma mL), distribution of antiretroviral drugs across diverse cellular and anatomic compartments in vivo is unequal. This leads to the acquisition by HIV of resistance to all known classes of currently prescribed retroviral drugs and to the establishment of viral reservoirs in vivo. Therefore, HIV has a distinct advantage of surviving in the host via a pre- and postintegration latency. Postintegration latency is caused by inert and metabolically inactive proviruses, which are not accessible neither by the immune system nor the drugs, and this state provides HIV with a safe haven in the host cell. With suitable stimuli it is possible to rekindle a viral latency stage, however, even though this mechanism has been described at least since 2000, the HIV reservoir existence remains to date the biggest impediment to its eradication in the human body. The existence of this latent reservoir and its features of extreme stability make the hopes of eradicating the virus through an antiretroviral therapy irrealistic. Therefore, the need to identify novel drugs against HIV infections, effective in the eradication of the virus from reservoirs in which it conceals and non-toxic, or anyhow with a lesser toxicity with respect to currently known antivirals, and possibly not capable of inducing pharmacoresistance phenomena, is markedly felt.

### SUMMARY OF THE INVENTION

In the present application there are reported for the first time three proline-rich peptides, two of salivary origin and one of synthetic origin but of salivary derivation, which, besides inhibiting viral infection in vitro without inducing toxic effects on mononucleard cells of peripheral blood, are able to cause temporary stimulation of viral replication.

The peptides of the present invention are proline-rich peptides with anti-HIV-1 activity. Two peptides were isolated, respectively from human saliva (SEQ ID NO 1, molecular weight of peptide: 1932 Da), and from pig saliva (SEQ ID NO 2, molecular weight of peptide: 2733 Da). A third peptide of SEQ ID NO 3, of molecular weight 1413 Da, is a variant not found in nature of a porcine salivary peptide, of 1511 Da, isolated from secretory granules of pig and present in the sequence of basic-proline-rich protein Q95JC9 of Sus scrofa. The structure of the peptide of SEQ ID NO 1, of 1932 Da, is present also in a repetitive way in the sequence of the two human proproteins Basic salivary proline-rich protein 1 and Basic salivary proline-rich protein 2 (Alternative name: Con1 glycoprotein). The same structure is also contained in sequence 753-768 of Merozoite surface protein-1 [msp1] [Plasmodium coatneyi]. The sequence SEQ ID NO 2, of the 2733 Da-peptide is present in the structure of isoforms 2 and 3 of the basic proline-rich protein from Sus scrofa (Q95JC9-2, -3). The 1932 Da- and 2733 Da-peptides were originally isolated and purified chromatographically, respectively from whole human saliva (mainly of parotid secretion) and from secretion granules isolated from pig parotid glands. The peptide of SEQ ID NO 3, of 1413 Da, is instead a synthetic product. All three peptides are characterized by a high content of proline residues: 45% (SEQ ID NO 1, 1932 Da), 68% (SEQ ID NO 2, 2733 Da) and 75% (SEQ ID NO 3, 1413 Da) and exhibit a markedly basic character. All three peptides, studied by circular dichroism and infrared spectroscopy, have a secondary structure characterized by the presence of a mixed structure comprised of a random coil pattern and polyproline-II, the latter more evident as temperature decreases. These structural features (proline content and secondary structure) suggest their potential as putative interactors with protein domains of SH3, WW, GYF, EVH1 type, as also demonstrated by “in silico” experiments using iSPOT online software (http://cbm.bio.uniroma2.it/ispot/).

The peptides described were evaluated for inhibitory activity against HIV-1 replication in experimental models of ex vivo endogenous and in vitro exogenous infection. The ex vivo endogenous infection model envisages the cultivation of peripheral blood mononucleard cells (PBMCs) collected from R5-HIV-positive subjects, in acute stage of infection and characterized by a high viral load at the plasma level, stimulated every 4 days with recombinant IL-2 and treated with the peptides of interest.

They showed the surprising and unexpected ability not only to inhibit viral replication in in vitro-infected cells, but also to induce ex vivo replication in cells collected from HIV-infected individuals.

As will be seen in more detail below, this ability to induce virus replication in cells in which it is present in a latent form, and to successively inhibit its replication (after having stimulated an initial replication of the latent virus), indicates the ability of these peptides to attack viral reservoirs and denotes them as active principles for the treatment of HIV infection and its eradication in the human body. This activity is described for the first time in the present invention and represents the first effective medical approach for the eradication of HIV virus, as is evident from recent works on HIV reservoirs reported in the literature, and mentioned in the foregoing, at the end of 2010.

Moreover, the peptides of the present invention exhibit a very low cytotoxicity, making them suitable for medical (therapeutic) use, and an antifungal activity particularly useful in the treatment of HIV infection and/or in therapy for AIDS, since oft-times patients suffering from immune deficiency are attacked by highly pathogenic opportunistic fungi (data reported in the experimental section below).

Besides the absence of cytotoxicity, which per se already makes the peptides described herein particularly suitable for medical use, the peptides of SEQ ID NO 1, 2 and 3 exhibit very high stability, probably due to their salivary origin (as it is known, in saliva there are several proteasic activities processing numerous proteins: therefore the peptides of the invention have already undergone the most recurrent processings performed by salivary proteases); moreover, the presence of a high number of proline residues is another protective factor against the most common proteases. Data reported in the experimental section below demonstrate that the activity of peptides placed in a cell culture medium remains unaltered for at least 4 days.

The stability of the molecules claimed herein is a feature particularly interesting in molecules for medical use, as it allows to limit the number of administrations. Moreover, their salivary derivation, and the processings already undergone by the same from salivary proteolytic enzymes, make them particularly stable and suitable even for oral administration.

Finally, in preliminary experimental data reported in the experimental section the peptides of SEQ ID NO 1, 2 and 3 also exhibit a good antimycotic activity, tested in particular on fungi particularly aggressive and commonly active in immunocompromised patients, such as patients suffering from AIDS.

Therefore, the peptides described herein exhibit an exceptional set of features which make them particularly suitable for medical use in the therapy against HIV infections, for eradicating HIV and in therapy for AIDS.

Specifically, it is exceptional the feature, first described herein, of peptides of SEQ ID NO 1, 2 and 3 being “searchers and destroyers” of HIV reservoirs.

The features of the peptides reported herein are absolutely individual and not ascribable to peptides exhibiting the general features reported above. In fact, together with peptides of SEQ ID NO 1, 2, and 3, alike peptides were tested, having a similar composition and even fragments thereof, which did not exhibit the features of the three peptides listed above.

The peptide of SEQ ID NO 1 and three further peptides, fragments or derivatives thereof (SEQ ID 5, 6, 7) were analyzed particularly for inhibition of HIV replication, as they contained a sequence (SEQ ID NO 4) which, according to the prior state of the art, should have conferred thereto alpha-glucosidase inhibition activities effective in HIV inhibition.

Also other two peptides fragments of the peptide of SEQ ID NO 1, i.e. the peptides of SEQ ID NO 8 and 9, and other four peptides having a composition similar to the peptides of SEQ ID NO 1, 2 and 3, i.e. the peptides of SEQ ID NO 10, 11, 12 and 13, were analyzed.

The data obtained are reported in the experimental section.

Despite structural analogies, the peptides of SEQ ID NO 1, 2 and 3 have some distinctive aspects:

A) the peptides of SEQ ID NO 1, 2 and 3 are able to activate the HIV-1 virus in ex vivo replication assays, an activity which, as will be described hereinafter, entails applicative aspects of remarkable medical (therapeutic) interest;

B) even though a direct comparison is not possible, it seems however evident that the peptides of SEQ ID NO 1, 2 and 3, though used at slightly higher concentrations, between 1.4 and 14 times those described in U.S. Pat. No. 5,981,720 for Con1 (no change), have an inhibitory activity 130 times higher than Con1, with values which can be overlapped to the control;

C) even though the peptides of SEQ ID NO 1, 5, 6 and 7 contain SEQ ID NO 4 GGNK, accountable, according to U.S. Pat. No. 5,981,720, for the blocking of HIV-1 infection through alpha glucosidase inhibition, data obtained from the experiments carried out clearly contradict this thesis. In fact, the peptides of SEQ ID 5, 6 and 7, which are fragments or derivatives of the peptide of SEQ ID NO 1 and contain SEQ ID NO 4, in no way influence the expression level of the viral antigen p24, and exhibit none of the activities listed in points A), B) and C).

Also the other 6 peptides assayed, of SEQ ID NO 8, 9, 10, 11, 12 and 13, do not exhibit any of the activities listed at A), B) and C).

Without wishing to bind the invention to the hypotheses formulated by the Inventors on the basis of obtained data, as to what underlies the inhibitory activity of the three peptides on HIV-1 virus proliferation mainly two hypotheses can be advanced. As already reported in two studies (U.S. Pat. No. 5,981,720, and White M R et al 2009), it is possible that some proline-rich peptides and proteins may have an inhibitory activity on glycosidic enzymes. In fact, both in the first and in the second study there are reported inhibition data by salivary components towards alpha glucosidase and Neuraminidase, enzymes required for proliferation respectively of the HIV-1 and influenza viruses. However, it is not apparent which other features said peptides should possess in order to have inhibitory activity on HIV proliferation.

The second hypothesis of mechanism (to be verified, as it is not assessable a priori or on the mere basis of the sequence), might lie in the potential ability of the three peptides of SEQ ID NO 1, 2 and 3, of interacting with SH3-type protein domains, able to recognize peptide sequences bearing the consensus motif PXXP (SEQ ID NO 14) (where X stands for any amino acid). This hypothesis is supported by the Stangler T study of 2007, in which it is described that in the HIV-1 proliferation mechanism the interaction between the viral Nef protein and the kinase Hck present on CD4+ cell membrane performs a key role. Said interaction occurs between a proline-rich Nef sequence and the SH3 domain present on the kinase; the peptides of the invention might interfere with this interaction, actually preventing viral proliferation.

A third hypothesis of mechanism is based on the work by Yang (Yang B e coll. 2003) in which it is demonstrated how some peptide sequences of 12 and 15 proline-containing residues are able to inhibit in vitro Vif (Viral infectivity factor) protein multimerization. Vif is essential for in vivo replication of (HIV-1) virus, and the Authors demonstrated that Vif proteins are able to form multimers, among which dimers, trimers, tetramers. Since the multimerization of Vif proteins is required for Vif function in the viral life cycle, this protein is proposed as a novel target for anti-HIV-1 therapeutics, and proline-rich peptides represent putative anti-HIV agents.

The present invention specifically relates to the peptide of SEQ ID NO 1.

This peptide exhibits antiviral activity towards the HIV virus and is also able to activate virus replication from viral reservoirs that said virus is able to create in the host organism.

The peptide of SEQ ID NO 1 could be used as sole active principle, or it could be used in combination with one or both of the peptides of SEQ ID NO 2 and 3 (exhibiting the same fundamental features, indicated above, of the peptide of SEQ ID NO 1) and/or with other drugs commonly used in therapy against HIV infections and/or in therapy for AIDS.

Therefore, object of the invention are a peptide of SEQ ID NO 1 with antiviral and antimycotic activity, nucleotide sequences coding for said polypeptide, a pharmaceutical composition comprising an antiviral peptide of SEQ ID NO 1 as active principle and a pharmaceutically acceptable carrier; a medical treatment for the treatment of HIV infections and/or in the therapy for AIDS, comprising the step of administering, to a patient in need thereof, a pharmaceutical composition comprising an antiviral peptide of SEQ ID NO 1 as active principle and a pharmaceutically acceptable carrier in a pharmacologically effective dose of said composition; a pharmaceutical kit for concomitant or sequential administration, comprising one or more aliquots of the antiviral peptide of SEQ ID NO 1 as active principle in a pharmaceutically acceptable carrier and one or more aliquots of one or more of the active principles selected in the group: peptide of SEQ ID NO 2, peptide of SEQ ID NO 3 and optionally one or more other active principles commonly used in anti-HIV therapy.

### DETAILED DESCRIPTION OF THE FIGURES

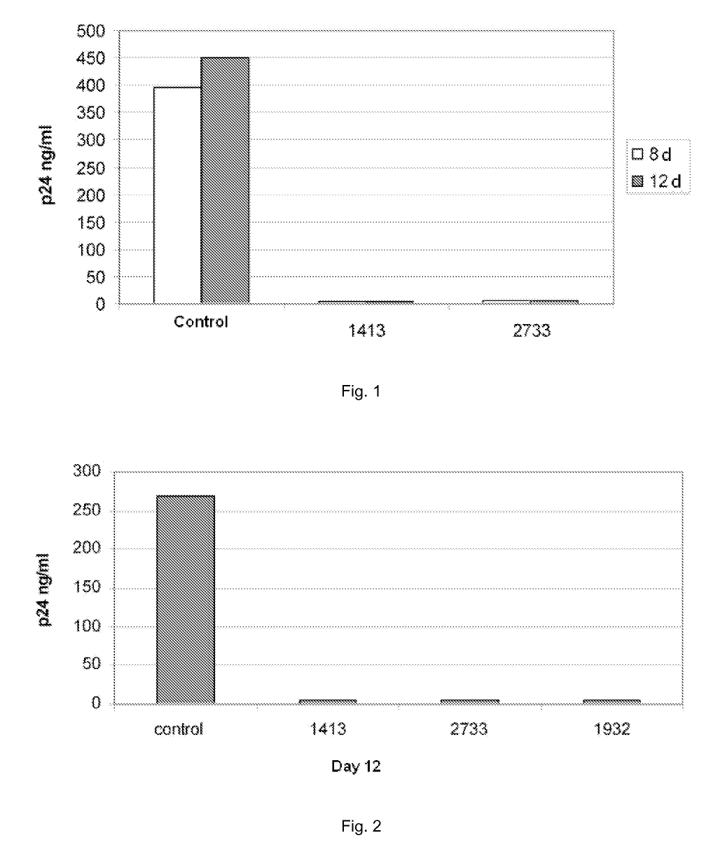

FIG. 1. Antiretroviral activity of peptides 1413 (SEQ ID NO 3) and 2733 (SEQ ID NO 2) in the in vitro assay of exogenous infection with HIV strain IIIB. The peptides were assayed at a concentration of 1 and 10 microg/mL (same results, having assessed the maximum concentration to exclude cytotoxicity phenomena). Dosage of p24 antigen, which is a protein present in the core of HIV-1 virus, i.e. in its internal part, is performed on cell culture supernatants at preset times; antigen presence in the culture broth is indicative of a high viral replication: In vivo, p24 Ag presence is detectable in the period immediately following contagion and in the advanced stages of the disease. In the figure it is evident how, in the presence of peptides 1413 (SEQ ID NO 3) and 2733 (SEQ ID NO 2) at the concentration of 10 microg/ml, virus replication over the time considered is almost equal to zero.

FIG. 2. Antiretroviral activity of peptides 1413 (SEQ ID NO 3), 2733 (SEQ ID NO 2) and 1932 (SEQ ID NO 1) at the concentration of 10 microg/ml, in the in vitro assay of exogenous infection with the HIV strain BaL. Like in FIG. 1, the amount of p24 antigen was measured in this assay as well. In particular, this analysis was carried out after 12 days and is indicative of a long-term effect of the same peptides.

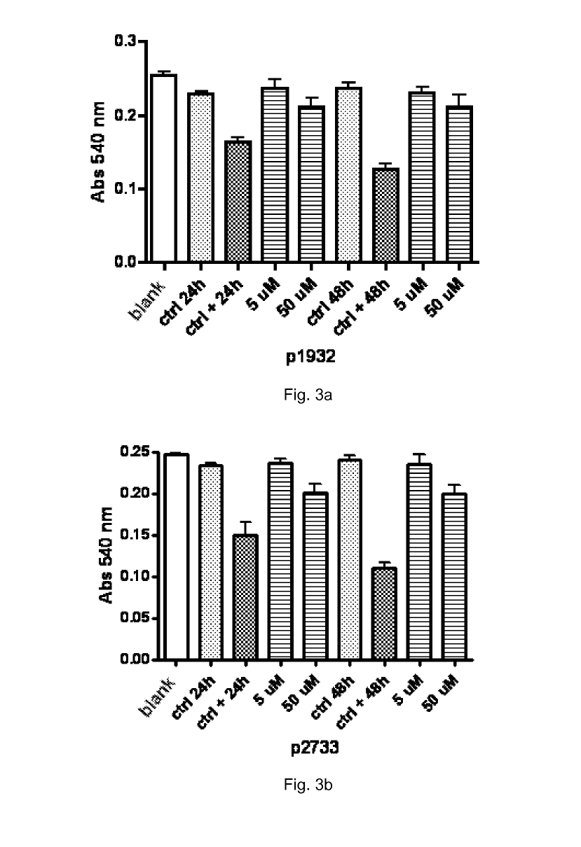

FIG. 3 (a, b, c). Cytotoxicity tests according to Neutral Red Uptake (NRU) method, performed on the three peptides: 1932 (SEQ ID NO 1), 2733 (SEQ ID NO 2) and 1413 (SEQ ID NO 3), at the two concentrations of 5 and 50 microM at +24 and +48 hours from administration. Therefore, concentrations near (5 microM) to or markedly higher (50 microM) than those employed for carrying out the ex-vivo and in vitro tests were used. Cells used are a tumour cell line denominated PE/CA PJ15. Positive control (denoted in the legends by ctrl+), used at a sublethal concentration of 10 microM, is a commercial proapoptotic peptide, Catalog #62206 (Anaspec-USA). Among the three, peptides p2733 (SEQ ID NO 2) and p1932 (SEQ ID NO 1) are, at concentrations approximately 12 times higher (50 microM) than those of use, slightly more toxic with respect to the control, both at +24 and +48 hours from administration. The lack of toxicity revealed by this test highlights that, at least for the cell model used, the peptides at issue cause no damage to cell membranes, or cause a limited damage thereto, and only at high concentrations.

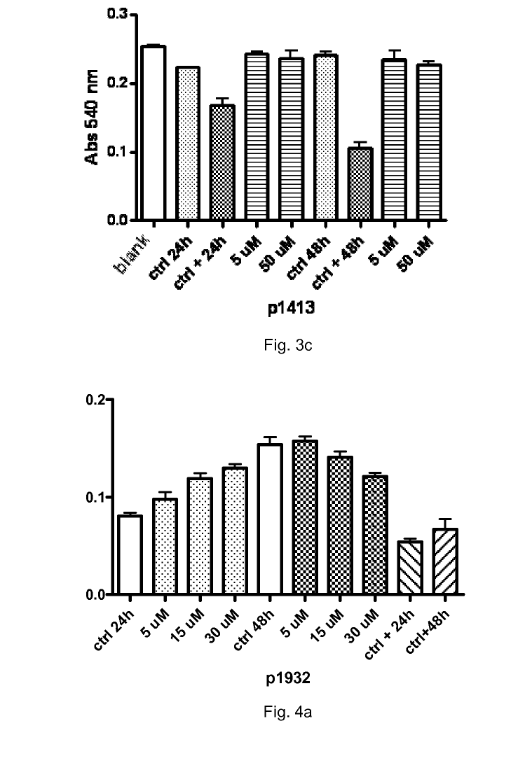

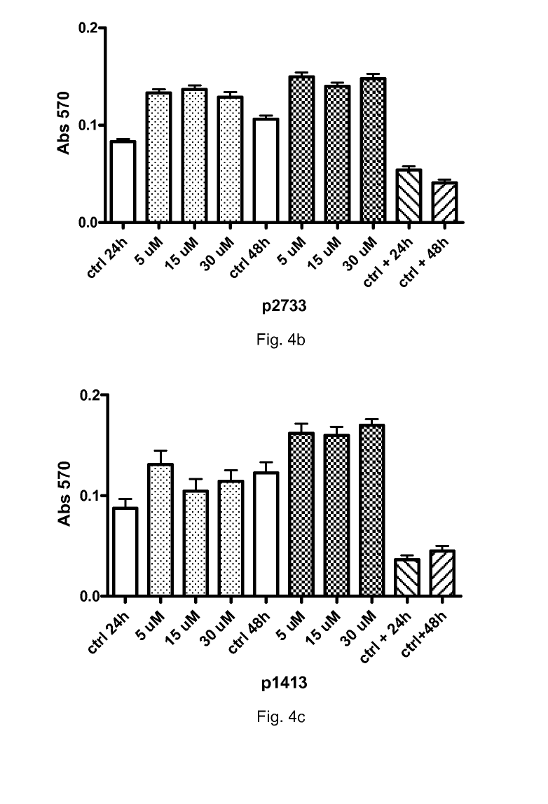

FIG. 4 (a, b, c). Cytotoxicity tests according to the MTT method (see protocols) performed on the three peptides: 1932 (SEQ ID NO 1); 2733 (SEQ ID NO 2) and 1413 (SEQ ID NO 3), and at three concentrations of 5, 15 and 30 microM at +24 and +48 hours from administration. There were used concentrations near (5 microM) to, or greater (15, 50 microM) than those used in the ex-vivo and in vitro tests, where average concentration of the three peptides is of about 3.65 microM. Cells used are a tumour cell line denominated PE/CA PJ15. Positive control (denoted in the legends by ctrl+), used at a sublethal concentration of 10 microM, is a commercial proapoptotic peptide, Catalog #62206 (Anaspec-USA). Peptide p1932 (SEQ ID NO 1) is slightly toxic with respect to the control at concentrations approximately 4 times higher (15 microM) than those of use, at +48 hours from administration. The lack of toxicity revealed by this test highlights that, at least for the cell model used, the peptides cause no damage at the mitochondrial level.

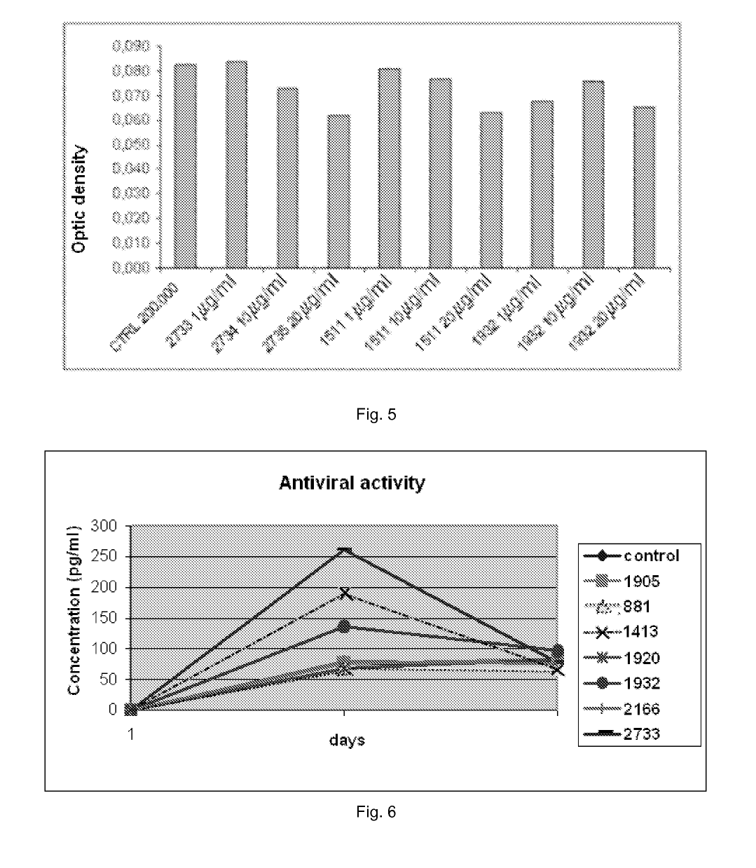

FIG. 5. Cytotoxicity test. The assay indicates the absence of significant cytotoxic effects at the concentrations used (1, 10, 20 microg/ml). In the assay performed, the high values of optical density indicate a high cell survival and a consequent low cytotoxicity.

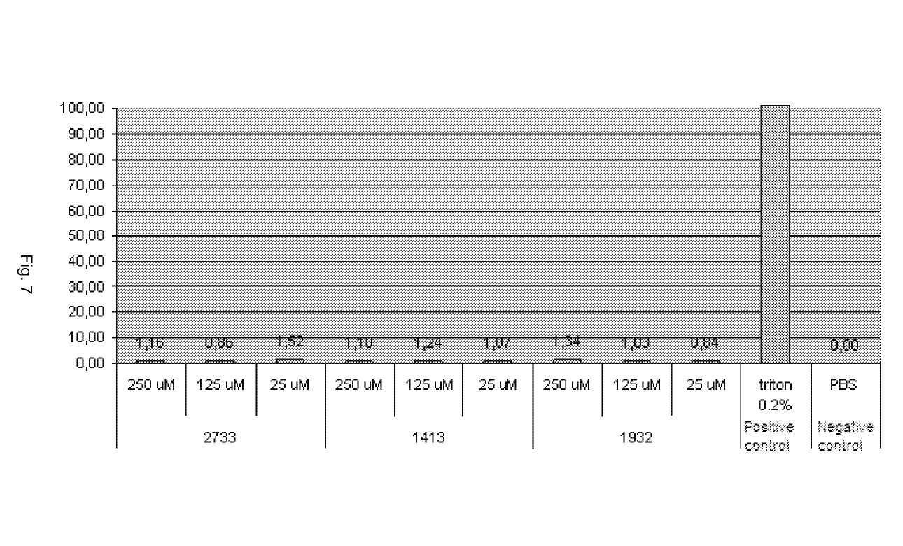

FIG. 6. Ex vivo endogenous replication assay. The test highlights the effects of peptides 1932 (SEQ ID NO 1), 2733 (SEQ ID NO 2) and 1413 (SEQ ID NO 3) on viral replication on day 5 of culture. p24 levels on day 10 of culture tend to decrease, like in the untreated control. In the figure, also results obtained from the treatment of cells with non-active peptides are shown (values similar to control).

FIG. 7. Graph representing preliminary toxicity results obtained via hemolysis experiment on human erythrocytes, to assess the cytotoxic effect of peptides of SEQ ID NO 1, 2 and 3 on erythrocytes and the consequent suitability thereof for administration through the bloodstream.

The graph therefore represents the result of a spectrophotometric test in which absorbance is read at 405 nm (corresponding to an absorption peak of hemoglobin). A cytotoxic activity of the peptides on the erythrocytes would lead to a lysis of the latter and to hemoglobin release in the suspension medium, spectrophotometrically quantifiable via an absorbancy measurement. In the graph, to a low percentage there corresponds a low cytotoxicity level.

100% hemolysis was obtained by using 0.2% Triton X-100 (positive control). Negative control is instead given by the suspension buffer (PBS).

### DESCRIPTION OF SEQUENCES

SEQ ID NO 1 Peptide derived from human saliva (molecular weight 1932 Da, in the text and figures also referred to as peptide 1932): GPPPQGGNKPQGPPPPGKPQ

SEQ ID NO 2 Peptide derived from secretion granules isolated from pig parotid gland (molecular weight 2733 Da, in the text and figures also referred to as peptide 2733): DKPKKKPPPPAGPPPPPPPPPGPPPPGP

SEQ ID NO 3 Synthetic peptide (molecular weight 1413 Da, in the text and figures also referred to as peptide 1413): RPPPGPPPPGPPPGP

SEQ ID NO 4 GGNK sequence, described in U.S. Pat. No. 5,981,720 as a sequence with inhibitory activity on viral alpha-glucosidase enzyme SEQ ID NO 5 “Peptide 1a”

Derivative of the peptide of SEQ ID NO 1: GPPPQGGNKPQGPPPPG

SEQ ID NO 6 “Peptide 1b”

Derivative of the peptide of SEQ ID NO 1: GPPPQGGNKPQ

SEQ ID NO 7 “Peptide 1c”

Derivative of the peptide of SEQ ID NO 1: GPPPQGGNKPQGPPPG

SEQ ID NO 8 “Peptide 1d”

Derivative of the peptide of SEQ ID NO 1: GPPPQGGN

SEQ ID NO 9 “Peptide 1e”

Derivative of the peptide of SEQ ID NO 1: GPPPPGKPQ

SEQ ID NO 10 “Peptide 4”—Peptide of porcine origin:

APPGARPPPGPPPPGPPPPGP

SEQ ID NO 11 “Peptide 5”—Peptide of porcine origin:

RSPFFDL

SEQ ID NO 12 “Peptide 6”—Peptide of porcine origin:

APPGARPPPPPPPPADQPQQGP

SEQ ID NO 13 “Peptide 7”—Peptide of porcine origin:

APPGARPLPGPPPPGPPPPGP

SEQ ID NO 17 Consensus motif of SH3-type protein domains

PXXP

where X denotes any amino acid.

### GLOSSARY

The peptide of SEQ ID NO 1 in the present description is also referred to as “peptide 1932”, “1932” or “p1932”.
The peptide of SEQ ID NO 2 in the present description is also referred to as “peptide 2733”, “2733” or “p2733”.
The peptide of SEQ ID NO 3 in the present description is also referred to as “peptide 1413”, “1413” or “p1413”.
By “viral reservoir” in the literature it is meant a cell type or an anatomic site in association with which a replication-competent form of the virus accumulates and persists with more stable kinetic properties than the main pool of actively replicating virus.
EC₅₀: EC₅₀ (median effective concentration) can be defined as the concentration able to produce, for a certain time of treatment, an incidence equal to the 50% of the effect that is to be analyzed as measure of toxicity. In the data reported in the present description, the effect analyzed is cell growth inhibition.

### DETAILED DESCRIPTION OF THE INVENTION

Hence, the present invention relates to a peptide of SEQ ID NO 1 with antiviral and antimycotic activity.

Specifically, the antiviral activity is performed against HIV virus, the etiological agent of human acquired immune deficiency syndrome.

In particular, as shown from data reported in the present description, the peptide of SEQ ID NO 1 (as well as the peptides of SEQ ID NO 2 and 3) shows a high antiviral activity and, surprisingly, an activity, observed in the ex vivo esperiments and never described before, consisting in the ability to induce replication of the HIV provirus integrated in peripheral blood lymphocytes, and the consequent applicability in the treatment aimed at the final eradication of the virus from the above-described reservoirs.

From an applicative standpoint all three peptides described can easily be synthesized, even without the aid of specific instrumentation, e.g. with the solid-phase synthesis technique using the F-moc (Fluorenyl-methyloxycarbonyl chloride) chemistry. This method, in case of proline-rich peptides, is exceedingly effective just because of the configuration that the peptides assume during the same synthesis. Incidentally, this fact allows high product yields with an entailed abatement of costs in view of a mass production.

The technician in the field could carry out the synthesis of the peptides described herein according to any one conventional technique and could obtain a pharmaceutical grade thereof with no need to exert inventive activity, even by placing an order, when desired, for the synthesis of one or more peptides to companies offering peptide synthesis among their services.

Therefore, the present invention also relates to nucleotide sequences coding for the peptides of SEQ ID 1, 2 and 3, which can be easily identified, even by the use of free software available to the technician in the field.

The sequences as meant herein are any nucleotide sequence coding for the peptides of SEQ ID 1, 2 and 3, taking into account the well-known genetic code degeneration. The genetic code is defined “degenerated”, as more codons can code for a same amino acid. Triplets coding for the same amino acid usually have the first two positions preserved, whereas the third position varies (e.g., amino acid Proline, recurring in the peptides described herein, can be coded for by codons CCA, CCC, CCG, CCU, CCT).

Such sequences could be used, e.g. inserted into suitable expression vectors known in all conventional techniques of genetics and molecular biology for the synthesis of the peptides of SEQ ID 1, 2 and 3.

The peptides of SEQ ID 1, 2 and 3 were evaluated for inhibition activity of HIV-1 replication in experimental models of ex vivo endogenous and in vitro exogenous infection. The ex vivo endogenous infection model (see FIG. 6 and experimental section below) envisages the cultivation of peripheral blood mononuclear cells (PBMCs) collected from HIV-R5 positive subjects, in acute stage of infection and characterized by high viral load at the plasma level, stimulated every 4 days with recombinant IL-2 and treated with the peptides of interest. The in vitro model utilizes PBMCs from healthy donors, stimulated beforehand with phytohemagglutinin (PHA) and rIL-2 and infected with HIV-1 strains X4 (IIIB) or R5 (BaL) according to two different protocols, respectively envisaging i) a single treatment of the cells with the peptides of interest at the concentration of 10 μg/ml before the infection and the stimulation with rIL-2 every 4 days; ii) cell infection with HIV-1 and successive treatment with the peptides of interest (10 μg/ml) and rIL-2 every four days. Production of viral antigen p24, quantified by immunoenzymatic assay (ELISA, Enzyme-Linked Immunosorbent Assay, Perkin Elmer ultrasensitive) in the culture supernatant of treated and control cells, was used as parameter of biological activity. In the ex vivo assay the treatment with the three peptides induced, after 5 days, a significant increase in viral replication in comparison with values detected in the controls (see Examples section). Thereafter viral replication tended to decrease, until reaching, on day 10, values similar to the controls (see Examples section). This temporary stimulation of viral replication is particularly interesting, since substances able to activate viral replication have been proposed as therapy adjuvants due to their ability to prevent the establishment of latent reservoirs or to eliminate the pre-existing ones and allow the final eradication of the virus from the body. In the in vitro assay (see FIGS. 1 and 2), performed by infecting PBMCs from healthy donors with the HIV strain IIIB, the peptides of SEQ ID 1, 2 and 3 exhibited instead a relevant effect of inhibition of viral replication (see Examples section for the data).

Therefore, the peptide of SEQ ID NO 1, optionally in combination with one or both of the peptides of SEQ ID NO 2 and 3, is particularly suitable for use in a medical treatment, in particular, for use in the treatment and/or prevention of HIV infection and/or for the eradication of HIV virus from viral reservoirs in the patient and/or in the therapy for AIDS.

A particular use of the peptide of SEQ ID NO 1 alone or in combination with one or both of the peptides of SEQ ID NO 2 and 3 and, optionally, with one or more further active principles as indicated above, is in the eradication treatment of HIV reservoirs.

The anti-reservoir functions exhibited by the peptides of SEQ ID 1, 2 and 3 in ex vivo experiments indicate the latter as active principles able to stimulate replication of the latent virus and, subsequently, to effectively inhibit its replication.

Therefore, the peptides described herein are able to bring the virus out of the reservoirs in which it nests, and to subsequently inhibit its replication, thereby leading to eradication of the virus from the HIV patient.

Hence, are part of the present invention: a use of the peptide of SEQ ID NO 1 alone or in combination with one or both of the peptides of SEQ ID NO 2 and 3 in a therapeutic method for the eradication of HIV reservoirs and/or for the treatment and/or the prevention of HIV infections and/or in the therapy for AIDS, comprising the step of administering to a patient in need thereof a pharmacologically effective dose of said peptide.

The therapeutic method according to the present invention, for the eradication of HIV reservoirs and/or for the treatment and/or the prevention of HIV infections and/or in the therapy for AIDS, comprises the step of administering to a patient in need thereof a pharmacologically effective dose of said peptide alone or in combination with one or both of the peptides of SEQ ID NO 2 and 3. Such administration could be repeated plural times and in plural administration cycles.

The amount of peptide of SEQ ID NO 1, alone or in combination with one or both of the peptides of SEQ ID NO 2 and 3, could be preset in unitary daily doses, or at intervals of plural days or weeks, or it could be evaluated by the medical staff according to the patient's disease stage, weight, gender and age.

By “therapeutically effective dose” it is meant a dose allowing the researcher or the physician to observe the desired therapeutic effect in the treated patient. Specifically, a therapeutically effective dose will be a dose (administered in one or more unitary doses (dosages) over time) leading to a partial or total reduction of HIV presence in the treated patient.

The therapeutically effective dose could be, as indicated above, administered in one or more unitary doses, and peptide administration could concomitantly or sequentially be associated with one or both of the peptides of SEQ ID NO 2 and 3 and/or with one or more active principles or drugs commonly used in the treatment of HIV infections and/or in the therapy for AIDS, such as antivirals, antimycotics, antibacterials as described in the present application or commonly known to a technician in the field.

In particular, the data reported in the present description show that in the peptides of SEQ ID NO 1, 2 and 3, the antiviral activity is accompanied by the antifungal one; this represents an added value in the field of the fight against AIDS, where disease progression is characterized by the onset of various opportunistic infections. The results described were confirmed in all experiments conducted by following the two different protocols of cell infection and treatment.

A table showing the antifungal activity of the peptides described herein is reported in the experimental section.

Moreover, data obtained from the above-described experiments demonstrate that the peptides of SEQ ID NO 1, 2 and 3 are more stable than current drugs used in the therapy of AIDS and anti-HIV treatments, which require daily administration. In fact, results obtained on cells treated with each peptide before infection are equivalent to those obtained by treating the cells with each peptide after infection, demonstrating also that the peptides of the invention remain functional for at least four days, whereas current drugs used in the therapy for AIDS and in the anti-HIV therapies are daily-dosage ones.

A further important feature of the three peptides is the absence of cytotoxic effects (see FIGS. 3, 4 and 5). While this aspect may be expected for human peptide 1932, it is not as expected for the peptide of porcine origin, nor for that of synthetic origin. The three peptides have been tested on cell line PE/CA PJ15, a squamous carcinoma of the tongue, without highlighting cytotoxic effects (see Examples section for the data). Moreover, from cytotoxicity tests performed preliminarily to the evaluation of antiretroviral activity, it emerged that the three peptides exert no toxic effect on human PBMCs, nor on human erythrocytes. This is an aspect of primary importance for the medical use of the peptides at issue.

The present invention also relates to a pharmaceutical composition comprising an antiviral peptide of SEQ ID NO 1 as active principle and a pharmaceutically acceptable carrier.

The peptide of the invention, together with a conventionally used adjuvant, carrier, diluent or excipient, may be set in form of pharmaceutical compositions and unitary doses thereof, and in such forms it may be used as a solid, powder, liquid, semiliquid, on media and the like. Such pharmaceutical compositions and their unitary dose forms may comprise ingredients in conventional proportions, with or without additional active compounds such as, e.g., one or both of the peptides of SEQ ID NO 2 and 3 and/or other active principles commonly used in anti-AIDS therapy. By “unitary dose” it is meant the dose suitable for reaching the therapeutically effective dosage as defined above, and it is normally established also on the basis of the patient's age, weight, gender and health conditions.

The unitary dose could vary depending on the administration regimen selected by the physician. Evidently, the administration regimen should be suitable for achieving the desired therapeutic effect.

When used as drugs, the peptides of this invention are typically administered in the form of a pharmaceutical composition. Such compositions can be prepared in a way well-known in the pharmaceutical field and comprise at least the active ingredient represented by the peptide of SEQ ID NO 1 and a pharmaceutically acceptable carrier, optionally in combination with further excipients or compounds commonly used in the formulation of pharmaceutical compositions. Such compositions could further comprise one or both of the peptides of SEQ ID NO 2 and 3 and/or other active principles commonly used in anti-HIV therapy and in therapy for AIDS.

In general, the compounds of this invention are administered in a pharmaceutically acceptable amount. The amount of compound actually administered will typically be determined by a physician, in light of the relevant circumstances, including the condition to be treated, the administration route selected, the compound actually administered, the individual patient's age, weight and response, the severity of the patient's symptoms and the like.

As described above, the pharmaceutically acceptable compositions of the present invention further comprise a pharmaceutically acceptable carrier, adjuvant or vehicle that, as used herein, includes any and all solvents, diluents or other liquid vehicles, aiding agents for dispersion or suspension, surface active agents, isotonic agents, thickening or emulsifying agents, preservatives, solid binders, lubricants and the like, as suitable for the particular form of dose desired. Remington's Pharmaceutical Sciences, 16^{th} Ed., E. W. Martin (Mack Publishing Co., Easton, Pa., 1980) describes various carriers used in the formulation of pharmaceutically acceptable compositions and the known techniques for their preparation.

Unless a carrier or other compound is incompatible with the peptide of SEQ ID NO 1 or peptides of SEQ ID NO 2 or 3, e.g. because it produces any one undesired biological effect or harmfully interacts with any other component of the pharmaceutically acceptable composition, its use is envisaged in the scope of this invention.

Some exemplary materials that may serve as pharmaceutically acceptable carriers include, but are not limited to, ion exchangers, aluminium, aluminium stearate, lecithin, serum proteins such as human serum albumine, buffer substances (phosphate, sorbic acid or potassium sorbate), partial mixtures of glycerids of vegetable saturated fatty acids, water, salt or electrolytes such as protamine sulphate, disodium hydrogen phosphate, potassium hydrogen phosphate, sodium chloride, zinc salts, colloidal silica, magnesium trisilicate, polyvinylpyrrolidone, polyacrylates, waxes, fatty tissue, sugars, such as lactose, glucose and sucrose; starches, such as corn starch and potato starch; cellulose and derivatives thereof, such as sodium carboxymethyl cellulose, ethyl cellulose and cellulose acetate; tragacanth powder; malt; gelatin; talc; excipients, such as coca butter and suppository wax, oils, such as peanut oil, cottonseed oil, sunflower oil, sesame oil, olive oil, corn oil and soybean oil; glycols, such as propylene glycol or polyethylene glycol; esters, such as ethyl oleate and ethyl laurate; agar; buffering agents, such as magnesium hydroxide and aluminium hydroxide, alginic acid, pyrogen-free water, isotonic saline, Ringer's solution; ethyl alcohol and phosphate-buffered salines, as well as other compatible non-toxic lubricants, such as sodium lauryl sulphate and magnesium stearate, as well as coloring agents, releasing agents, coating agents, sweeteners, flavors and perfuming agents, preservatives and antioxidants, may also be present in the composition according to the judgment of who prepares the formulation.

The pharmaceutically acceptable compositions of this invention may be administered to human beings for (by) oral, parenteral, intravenous, aerosol, rectal, trasdermic, subcutaneous, intracisternal, intramuscolar, intravaginal, intraperitoneal, topical, perilingual and intranasal use (route) and according to all administration routes known to a technician in the field.

In certain embodiments, the peptide of SEQ ID NO 1 of the invention could be administered at dose levels of from about 0.01 mg/kg to about 50 mg/kg, e.g. from about 1 mg/kg to about 25 mg/kg of the subject's body weight per day, once or more per day, to obtain the desired therapeutic effect.

However, doses higher or lower than those indicated above are not excluded from the scope of the present invention.

The compositions for oral administration may take the form of liquid solutions or suspensions, or powders. The compositions could be presented in the form of unitary dose, so as to facilitate dosage.

The term “form of unitary dose” refers to a discrete physical unit suitable for unitary doses for human subjects, each unitary dose containing a predetermined amount of active material calculated for producing the desired therapeutic effect, in association with a suitable pharmaceutical excipient. Typical forms of unitary doses include vials or prefilled or premetered syringes of the liquid composition, or pills, tablets, capsules or the like in case of solid compositions and transdermal patches. In such compositions, the peptide of SEQ ID NO 1 represents from about 0.01 to about 51% by weight of the composition, with the remainder being represented by any other active principles and various vehicles or carriers useful to make the desired form of dose.

Liquid forms suitable for oral administration include, but are not limited to, pharmaceutically acceptable emulsions, gels, microemulsions, solutions, suspensions, syrups and elixirs, and could contain diluents commonly used in the state of the art. The oral liquid forms can therefore include a suitable aqueous or non-aqueous vehicle with buffers, aiding agents for suspension and dispersion, emulsifying agents, solvents, colorants, flavors and the like.

E.g., they could include water or other solvents, solubilizing and emulsifying agents such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, (wheat) germ, olive, castor and sesame oils), glycerol, tetrahydrofurfuryl acid, polyethylene glycols and fatty acid esters of sorbitan and mixtures thereof. Besides inert diluents, the oral compositions could also include adjuvants such as humectants, emulsifying and suspending agents, sweetening, flavoring and parfuming agents.

The salivary origin or derivation of the peptides described herein makes a further degradation thereof when orally administered unlikely.

The liquid forms could also be injectable preparations, e.g. sterile injectable aqueous or oleaginous suspensions, and could be formulated according to the known art by using suitable dispersing agents, humectants and suspending agents. The sterile injectable preparations could also be an injectable sterile solution, suspension or emulsion in an parenterally acceptable atoxic diluent or solvent, like, e.g., a solution in 1,3-butanediol. Among the acceptable vehicles and solvents that may be used there are water, Ringer's solution, USP and isotonic sodium chloride solution. Furthermore, sterile fixed oils are conventionally used as solvent or suspending means. Moreover, fatty acids like oleic acid are used in injectable preparations.

Preservatives and other additives such as antimicrobials, antioxidants, chelating agents and inert gases could also be present (Mack (1982) Remington's Pharmaceutical Sciences, 16^{th} Edition).

The injectable formulations could be sterilized, e.g., by filtration through a bacteria-retaining filter, or by incorporating sterilizing agents in the form of sterile solid compositions that may be dissolved or dispersed in sterile water or other injectable sterile means before use.

In order to prolong the effect of the peptide described herein in the case of injectable formulations, its absorption can be slowed down, e.g., by dissolving or suspending the peptide in an oily vehicle. Injectable slow-release formulations can be made also by forming microincapsulated matrices of the active principle(s) in biodegradable polymers such as polylactide-polyglycolide. Depending on the active principle(s)/polymer ratio and the nature of the specific polymer used, release rate can be controlled. Examples of other biodegradable polymers include poly(orthoesters) and poly(anhydrides). Injectable slow-release formulations can also be prepared by trapping the active principle(s) in liposomes or microemulsions compatible with body tissues.

Semiliquid (waxy) formulations. The compositions for rectal or vaginal administration are preferably suppositories that can be prepared by mixing the compounds of this invention with suitable non-irritating excipients or carriers such as cocoa butter, polyethylene glycol, or a suppository wax which is solid at room temperature but liquid at body temperature and therefore dissolves in the rectum or in the vaginal cavity, releasing the active compound.

Solid-dose forms for oral administration include capsules, tablets, pills, powders and granules. In such solid forms the active principle(s) is/are mixed with at least one pharmaceutically acceptable inert excipient or carrier, like, e.g., sodium citrate or calcium phosphate citrate and/or fillers or extenders (such as starches, lactose, sucrose, glucose, mannitol, and silicic acid) binders; (such as, e.g., carboxymethylcellulose, alginates, gelatin, polyvinylpyrrolidone, sucrose); humectants (e.g., glycerol); disintegrating agents (such as agar-agar, calcium carbonate, potato starch or tapioca starch, alginic acid, certain silicates and sodium carbonate); retarding agents (e.g., paraffin) absorption accelerators (such as quaternary ammonium compounds); wetting agents (such as, e.g., cetyl alcohol and glycerol monostearate); absorbents (such as kaolin and bentonite clay); lubricants (such as talc, calcium stearate, magnesium stearate, solid polyethylene glycols, sodium lauryl sulfate) agents and mixtures thereof.

In case of capsules, tablets and pills, the form of dose could also comprise buffering agents.

The solid compositions as indicated above can also be employed to fill capsules of rigid or soft gelatin using excipients like lactose or milk sugar, as well as high-molecular weight polyethylene glycols and the like. The solid dose forms of tablets, film-coated tablets, capsules, pills, and granules, can be prepared with coatings such as enteric coatings and other coating agents known in the art of pharmaceutical formulations. Optionally, they could be formulated so as to release the active ingredient or the active ingredients solely or preferentially in certain parts of the intestinal tract, optionally in a delayed manner.

Specifically, the salivary origin or derivation of the peptides described herein makes a further degradation thereof when orally administered unlikely.

The compositions of the present invention could also be formulated for topical administration in the form of ointments, pastes, lotions, gels, powders, solutions, sprays, inhalants, ophthalmic or optic drops, or plasters. The active component (or components) is mixed under sterile conditions with a pharmaceutically acceptable carrier and, needwise, with any required preservative or buffer.

The transdermal patches could be used to provide a controlled release. Absorption enhancers could also be used, to increase the flow of the compound through the skin. The release rate could be controlled by providing a rate-controlled membrane or dispersing the compound into a polymer matrix or a gel.

Therefore, as indicated above, the peptide of SEQ ID NO 1, optionally together with one or more further active principles, could also be administered in prolonged-release forms or with systems for administration of prolonged-release drugs. A description of materials for such embodiment can also be found in the materials incorporated in Remington's Pharmaceutical Sciences.

The above-described components for oral administration or injectable compositions are merely representative. Further materials, as well as processing techniques and the like are shown in Part 8 of Remington's Pharmaceutical Sciences, 17^{th} Edition, 1985, Marck Publishing Company, Easton, Pa., which is incorporated herein as reference.

The peptides of this invention can be lyophilized for storage and reconstituted in a suitable carrier before use. For this embodiment, lyophilizing and reconstituting techniques known in the state of the art can be used. Persons skilled in the art will realize that the lyophilizing and reconstituting may lead to various degrees of loss of activity, and that the levels of use should be upregolated in order to compensate therefor.

Merely by way of a non-limiting indication of the embodiment of the invention, the peptide of SEQ ID NO 1 in such compositions could be in the range of between 0.05 and 51%, e.g. between 0.05 and 10, 20, 30, or 40% by weight, with the remainder being injectable carrier and the like.

However, peptide percentages by weight different with respect to the total weight of the composition, higher, lower or comprised between those indicated above, are not excluded from the invention.

Generally, the peptide of SEQ ID NO 1 and in case one or both of the peptides of SEQ ID NO 2 and 3 will be used in a purified form of pharmaceutical grade, together with suitable pharmaceutical carriers.

As indicated above, the peptide of SEQ ID NO 1, optionally together with one or both of the peptides of SEQ ID NO 2 and 3, could be used as compositions to be administered separately or in conjunction with other therapeutic agents. These can include various immunotherapy drugs, such as cyclosporine, methotrexate, adriamycin or cisplatin and immunotoxins and/or one or more antiinflammatory drugs and/or one or more antibacterial drugs and/or one or more antimycotic drugs and/or other drugs commonly used in therapy for HIV.

In the composition of the invention, beside the peptide of SEQ ID NO 1 also other active principles could be present, like e.g. one or more of the active principles selected in the group: peptide of SEQ ID NO 2, peptide of SEQ ID NO 3, nucleoside inhibitors, non-nucleoside inhibitors, reverse transcriptase inhibitors, integrase inhibitors, viral protease inhibitors, virus-host cell fusion process inhibitors, antimycotics, antibacterials, and other active principles commonly used in the therapy for AIDS.

A non-limiting example of such active principles comprises:

the nucleoside inhibitors AZT, abacavir, stavudine; the non-nucleoside inhibitors nevirapine, efavirenz; reverse transcriptase inhibitors; the integrase inhibitors raltegravir; the viral protease inhibitors ritonavir, atazanavir, darunavir, nelfinavir;

antimycotics, such as, e.g. polyene antimycotics, like amphotericin B, nystatin; imidazole antimycotics, like miconazole, clotrimazole, econazole, ketoconazole, sulconazole, tioconazole; triazole antimycotics, like fluconazole, itraconazole, posaconazole, voriconazole;

echinocandins, such as Anidulafungin, Caspofungin;

flucytosine, griseofulvin;

antibacterials, such as first-, second- and third-generation cephalosporins;

virus-host cell fusion process inhibitors maraviroc, enfuvirtide and the like.

The concentration of the further one or more active principles in the composition could easily be established by persons skilled in the art on the basis of the concentrations normally used; usually it will be equal to or lower than, preferably lower than that commonly used in therapies with the further above-mentioned active principles.

In a preferred embodiment of the invention, said further one or more active principles will be present in the unitary dose of composition at concentrations lower than those commonly used in the therapy for HIV-infected patients and for AIDS.

The pharmaceutical compositions may include cocktails of various active principles indicated above, mixed or not mixed before administration.

The peptides of SEQ ID NO 1, 2 and/or 3 could be present in the pharmaceutical composition (and in the unitary dose to be used in the medical or preventive treatment of the invention) in alike or different weight percentages. In an embodiment, the peptide of SEQ ID NO 1 will be present in a weight percentage greater in the pharmaceutical composition than in the peptides of SEQ ID NO 2 and/or SEQ ID NO 3.

In an embodiment of the invention, the peptide of SEQ ID NO 1 will be present in the composition of the invention in a weight percentage greater with respect to each further active principle present in the composition, or it could be present also in a weight percentage greater than the overall weight of any and all other active principles present in the composition.

In an embodiment of the present invention, the peptide of SEQ ID NO 1 represents at least 51% by weight of the total active principles, both in formulations in which all of the active principles are mixed in a pharmaceutical composition and in embodiments in which the active principles are administered in different aliquots (see kit below) in a concomitant or sequential form.

The compositions containing the peptide of SEQ ID NO 1 or a cocktail comprising at least two between the peptides of SEQ ID NO 1, 2 and 3 (the peptide of SEQ ID NO 1 being one of the at least two peptides of the cocktail) can be administered for prophylactic and/or therapeutic treatments. Given the very low toxicity demonstrated by the peptides of the invention in the cytotoxicity experiments carried out, the above-indicated compositions could be used also for prevention by those subjects exposed to HIV infection, such as medical operators and partners of infected subjects.

Further, given the extreme stability of the peptides described herein, the unitary doses of the compositions of the invention could be administered at intervals of 1 to 7 days apart, e.g., every 1, 2, 3, 4, 5, 6, 7 days or more.

In an embodiment, the above-described compositions will comprise as sole active principle the peptide of SEQ ID NO 1.

Such compositions, given the anti-viral (anti-HIV), anti-HIV reservoir and anti-mycotic effect of the peptide of SEQ ID NO 1, and given its very low toxicity, could be used with success in the medical treatment of AIDS and of HIV seropositivity, as well as for a preventive treatment in order to nip in the bud any HIV infection whatsoever, and could be used, in particular, for the eradication of HIV reservoirs present in persons infected by said virus, allowing eradication of the disease.

In fact, as already mentioned, a particular use of the peptide of SEQ ID NO 1 alone or in combination with one or both of the peptides of SEQ ID NO 2 and 3 and, optionally with one or more further active principles as indicated above, is in the treatment for the eradication of HIV reservoirs.

The antifungal activity of the peptides of the invention (whose data are reported in the Examples section) makes them more suitable to use in the treatment of HIV infections which normally expose the immunocompromised individual to opportunistic infections by various pathogenic agents, fungi included. Preliminary data on the peptides of the invention show also a possible antibacterial activity that would be of evident usefulness in the treatment of immunocompromised patients.

The present invention also comprises a pharmaceutical kit for concomitant or sequential administration of the peptide or peptides of the invention, comprising one or more aliquots of the antiviral peptide of SEQ ID NO 1 as active principle and a pharmaceutically acceptable carrier and one or more aliquots of the one or more of the active principles selected in the group: peptide of SEQ ID NO 2, peptide of SEQ ID NO 3, nucleoside inhibitors such as AZT, abacavir, stavudine; non-nucleoside inhibitors, such as nevirapine, efavirenz; reverse transcriptase inhibitors; integrase inhibitors, such as raltegravir; viral protease inhibitors such as ritonavir, atazanavir, darunavir, nelfinavir; polyene antimycotics, such as amphotericin B, nystatin; imidazole antimycotics, such as miconazole, clotrimazole, econazole, ketoconazole, sulconazole and tioconazole; triazole antimycotics, such as fluconazole, itraconazole, posaconazole, voriconazole; echinocandins, such as Anidolafungin, Caspofungin; flucytosine, griseofulvin, antibacterials, such as first-, second- and third-generation cephalosporins; virus-host cell fusion process inhibitors, such as maraviroc, enfuvirtide and the like.

Each aliquot of the kit described herein could further comprise one or more of pharmaceutically acceptable carriers, adjuvants or vehicles that, as used herein, include any and all solvents, diluents or other liquid vehicles, aiding agents for dispersion or suspension, surface active agents, isotonic agents, thickening or emulsifying agents, preservatives, solid binders, lubricants and the like, as suitable for the specific dose form desired.

For the making of the aliquots, reference is made to the embodiments of the above-described pharmaceutical composition.

The kit could be formulated so as to be able to administrate said aliquots for oral, parenteral, intravenous, aerosol, rectal, transdermic, subcutaneous, intracisternal, intramuscular, intravaginal, intraperitoneal, topical, perilingual and intranasal use.

The active principles will be distributed in different aliquots to be administered concomitantly or sequentially. In an embodiment, an aliquot might contain the sole peptide of SEQ ID NO 1 and one or more other aliquots might contain one or more of the other active principles or drugs selected for the kit among the ones indicated above.

The pharmaceutical kit as described could be used in the prevention and/or in the treatment of HIV infection and/or for the eradication of HIV virus from viral reservoirs in individuals suffering from HIV and/or in therapy for AIDS.

Therefore, it could be used in a medical treatment for the prevention and/or the treatment of HIV infections and/or in the therapy for AIDS, comprising the step of administering concomitantly or sequentially to a patient in need thereof an aliquot comprising the antiviral peptide of SEQ ID NO 1 in a pharmaceutically acceptable carrier and one or more aliquots comprising one or more of the active principles selected in the group: peptide of SEQ ID NO 2, peptide of SEQ ID NO 3, nucleoside inhibitors; non-nucleoside inhibitors; reverse transcriptase inhibitors; integrase inhibitors; viral protease inhibitors; antimycotics; antibacterials; virus-host cell fusion process inhibitors.

All of what has been described above is also part of the embodiment of the present invention, which also relates to a method for the treatment and/or the prevention of HIV infection and/or for the eradication of HIV virus from viral reservoirs in individuals suffering from HIV and/or in therapy for AIDS.

The next section aims at illustrating the data obtained on the peptides of the present invention and the activities observed for the latter, and therefore aims at clarifying the invention without of course being limitative of the same.

### EXPERIMENTAL DATA AND EXAMPLES

The assays and experiments reported below were independently carried out on all three peptides of SEQ ID NO 1, 2 and 3. By way of example, sometimes assays on only one of the peptides of SEQ ID NO 1, 2 or 3 are reported, but the same have however been performed on the other two peptides as well.

1. Peptide Synthesis.

The peptides were assembled on an Applied Biosystems 433A Peptide Synthesizer (Foster City, Calif., USA) using the Fmoc protocol for the step-wise synthesis of solid phase peptides (1,2) on pre-loaded Proline or Glutamine-2-chlorotrityl resins (Novabiochem, Laufelfingen, CH) The Fmoc-(Nα-9-Fluorenylmethyoxycarbonyl) amino acids were obtained from Novabiochem. All attacks were performed with a 5-fold excess of activated amino acids in the presence of 10 equivalents of N-ethyldiisopropyl-amine, using N-[(dimethyllamino)-1-H-1,2,3-triazolo-[4,5-B]pyridino-1-ylmethylene]-N-methylmethanaminium hexafluorophosphate N-oxide (HATU, PE Biosystems, Inc., Warrington, UK) as activating agent for the carboxyl group. Removal of temporary Fmoc-protection of the amine group was obtained with (20%) piperidine in dimethylformamide. After completion of the peptide chain assembly, the peptide was cleaved from the resin by treatment with a mixture of 80% trifluoroacetic acid, 5% water, 5% phenol, 5% thioanisole, 2.5% ethanedithiol and 2.5% triisopropyl silane (reagent K, 3) for 3 hours at room temperature, with concomitant side chain deprotection. The resin was filtered and the peptide cold-precipitated in tert-butylmethyl ether. After centrifugation and washing with tert-butylmethyl ether, the peptide was suspended in 5% aqueous acetic acid and then lyophilized.

2. Peptide Purification

Analysis and semi preparative purification in reverse-phase high-performance liquid chromatography (RP-HPLC) was carried out by a Tri Rotar-VI HPLC system equipped with an MD-910 multi-channel detector for analytical purposes, or with a Uvidec-100-VI variable UV detector for preparative purpose (all from JASCO, Tokyo, Japan). Analytical RP-HPLC chromatography was carried out with a Jupiter 5μ C18 300A column (150×4.6 mm, Phenomenex, Torrance Calif., USA). Semipreparative RP-HPLC runs were carried out with a Jupiter 10μ C18 300A column (250×21.2 mm, Phenomenex, Torrance Calif., USA). A linear gradient of acetonitrile in water/0.1% TFA was used to elute the bound peptide. The eluate from the semipreparative runs was collected in fractions, and the fractions were evaluated by RP-HPLC analysis. MALDI-TOF (matrix-assisted laser desorption ionization/Time of Flight) mass analyses were performed on a Voyager-RP Biospectrometry Workstation (PE Biosystem, Inc.). Experimental values found for peptide masses were in accordance with theoretical calculated values.

1) Atherton E, Sheppard R C (1987) in Udenfriend S and Meienhofer J (eds) The Peptides vol. 9: 1-39, Academic Press, San Diego, Calif.

2) Fields G B, Noble R L (1990) Int J Peptide Protein Res 35: 161-214

3) King D S, Fields C G and Fields G B (1990) Int J Peptide Protein Res 42: 255-266.

3. List of Salivary Peptides Studied

| | |
|---|---|
| 1 | peptide of SEQ ID NO 1 |
| 2 | peptide of SEQ ID NO 2 |
| 3 | peptide of SEQ ID NO 3 |

The peptide of SEQ ID NO 1 and three further peptides, fragments or derivatives thereof (SEQ ID 5, 6, 7) were analyzed, particularly for inhibition of HIV replication, as they contained a sequence (SEQ ID NO 4) that, according to the state of the prior art, should have conferred thereto alpha glucosidase inhibition activities effective in HIV inhibition.

Also other two peptides, fragments of the peptide of SEQ ID NO 1 (SEQ ID 8 and 9) and other four peptides having a composition similar to the peptides of SEQ ID NO 1, 2 and 3 (SEQ ID NO 10, 11, 12 and 13) were analyzed.

TABLE 1

| | Name | Mr | sequence | Ip |
|---|---|---|---|---|
| 1 | 1932 | 1932 | SEQ ID 1 | 10.0 |
| | | | 1-GPPPQGGNKPQGPPPPGKPQ-20 | |
| 1a | 1-17 | 1578 | SEQ ID NO 5 | 8.75 |
| | | | 1-GPPPQGGNKPQGPPPPG-17 | |
| 1b | 1-11 | 1076 | SEQ ID NO 6 | 8.75 |
| | | | 1-GPPPQGGNKPQ-11 | |
| 1c | 1-17 | 1481 | SEQ ID NO 7 | 8.75 |
| | des | | 1-GPPPQGGNKPQGPPPG-16 | |
| | pro | | | |
| 1d | 1-8 | 722 | SEQ ID NO 8 | 5.52 |
| | | | 1-GPPPQGGN-8 | |
| 1e | 12-20 | 874 | SEQ ID NO 9 | 8.75 |
| | | | 12-GPPPPGKPQ-20 | |
| 2 | 2733 | 2733 | SEQ ID NO 2 | 10.0 |
| | | | DKPKKKPPPPAGPPPPPPPPPGPPPPGP | |
| 3 | 1413 | 1413 | SEQ ID NO 3 | 9.75 |
| | | | RPPPGPPPPGPPPGP | |
| 4 | 1905 | 1904 | SEQ ID NO 10 | 9.79 |
| | | | APPGARPPPGPPPPGPPPPGP | |
| 5 | 881 | 881 | SEQ ID NO 11 | 5.84 |
| | | | RSPFFDL | |
| 6 | 2166 | 2166 | SEQ ID NO 12 | 5.88 |
| | | | APPGARPPPPPPPPADQPQQGP | |
| 7 | 1920 | 1920 | SEQ ID NO 13 | 9.79 |
| | | | APPGARPLPGPPPPGPPPPGP | |

7 differently isolated salivary peptides and 5 derivatives of peptide 1, reported in Table 1 above (SEQ ID NO 1-3, 5-13) were studied. The general features in them are similar: all proline-rich and with a basic isoelectric point (Ip), save three of them (SEQ ID NO 8, SEQ ID NO 11 and SEQ ID NO 12). In vitro, with respect to the control, some peptides strongly inhibit HIV-1 replication (as demonstrated for the peptides of SEQ ID NO 1, 2 and 3); others are close to the replication levels of the controls.

The peptide of SEQ ID NO 1, activating (as well as the peptides of SEQ ID NO 2 and 3) HIV-1 replication in ex vivo experiments, contains the sequence GGNK (SEQ ID NO 4) accountable, according to U.S. Pat. No. 5,981,720, for the prevention of HIV-1 infection through alpha-glucosidase inhibition.

The peptide of SEQ ID NO 1 and three further peptides (SEQ ID 5, 6, 7), fragments or derivatives thereof were analyzed particularly for inhibition of HIV replication.

The experiments performed clearly belie the thesis reported in U.S. Pat. No. 5,981,720, as it was verified on subfragments of the same peptide (1a, 1b and 1c) that the presence of said sequence in no way influences the expression level of Ag p24. In terms of saliva peptides, therefore, not all of them possess an activity inhibiting HIV-1 replication though being proline-rich or possessing the GGNK sequence. In peptides 1, 2 and 3 (sequences SEQ ID NO 1, 2 and 3), the antiviral activity is summed to the antimicrobial activity on fungal strains (see reported data); in the field of the fight to progression of AIDS disease, where the onset of opportunistic infections is an actual fact, this represents an added value.

Also other peptides were analyzed, generated from variable and constant regions of immunoglobulins, some of which with antiviral, antibacterial and antitumoral activity (PLOS One, 2008). The inhibition index of the anti-HIV-1 activity thereof is markedly inferior (with respect to the controls, it is measured to be in the order of the 80%) to that demonstrated by the salivary peptides at issue (in the order of 3-4 Log).

4. Analysis of Antifungal Activity

Preliminary data were collected, reported in the following tables, related to the antifungal activity of peptides of SEQ ID NO 1, 2 and 3. As can be seen from Tables 2, 3 and 4, the peptides have a high antifungal activity even at medically (therapeutically) acceptable concentrations.

TABLE 2
Activity of peptide 1932 SEQ ID NO 1 1, expressed as inhibition
percentage of the growth of the corresponding fungal strain.

| peptide 1932 | | |
|---|---|---|
| concentration | Candida albicans | Cryptococcus neoformans |
| (μg/ml) | UP10 | 6995 |
| 100 | 41.17 | 85.82 |
| 50 | | 80.98 |
| 25 | | 66.18 |
| 12.5 | | |
| | | EC₅₀ 5.833 × 10⁻⁶ moles/liter |

TABLE 3
Activity of peptide 2733 SEQ ID NO 2 1, expressed as inhibition
percentage of the growth of the corresponding fungal strain.

| peptide 2733 | Candida | |
|---|---|---|
| concentration | albicans | Cryptococcus |
| (μg/mL) | UP10 | neoformans 6995 |
| 100 | 47.34 | 95.68 |
| 50 | | 94.1 |
| 25 | | 89.88 |
| 12.5 | | 74.15 |
| 6.25 | | 56.34 |
| 3.125 | | 22.44 |
| | | EC₅₀ 2.204 × 10⁻⁶ |
| | | moles/liter |

TABLE 4
Activity of peptide 1413 SEQ ID NO 3 1, expressed as inhibition
percentage of the growth of the corresponding fungal strain.

| peptide 1413 | | |
|---|---|---|
| concentration | Candida albicans | Cryptococcus |
| (μg/ml) | UP10 | neoformans 6995 |
| 100 | 31.09 | 84.81 |
| 50 | | 82.09 |
| 25 | | 52.40 |
| 12.5 | | 0 |
| | | EC₅₀ 1.764 × 10⁻⁵ |
| | | moles/liter |

From the data reported, it is evident how all three peptides are active to a different extent in inhibiting the growth of Cryptococcus neoformans, a fungus particularly virulent and opportunistic in case of patients suffering from immune deficiency.

In particular, peptide 1413 (SEQ ID NO 3) has an activity with an EC₅₀ equal to 17.64 microM, peptide 1932 (SEQ ID NO 1) with an EC₅₀ equal to 5.8 microM and peptide 2733 (SEQ ID NO 2) with an EC₅₀ equal to 2.2 microM, actually turning out to be the most effective one.

EC₅₀: EC₅₀ (median effective concentration) can be defined as the concentration able to produce, for a given treatment time, an incidence equal to the 50% of the effect selected as measure of toxicity (in this case, the effect followed is growth inhibition).

5. Ex Vivo and In Vivo Model of HIV Infection

The ex vivo endogenous infection model envisages the cultivation of peripheral blood mononuclear cells (PBMC) collected from HIV-R5 positive subjects, in acute stage of infection and characterized by a high viral load at the plasma level, stimulated every 4 days with recombinant IL-2 and treated with the peptides of interest at the concentration of 1-10 μg/ml.

The in vitro model utilizes PBMCs from healthy donors, stimulated beforehand with phytohemagglutinin (PHA) and rIL-2, and infected with HIV-1 strains X4 (IIIB) or R5 (BaL) according to two different protocols, respectively envisaging i) a single treatment of the cells with the peptides of interest at the concentration of 10 μg/ml before the infection and the stimulation with rIL-2 every four days; or, ii) cell infection with the virus and the successive treatment with the peptides of interest (10 μg/ml) and rIL-2 every four days.

Production of viral antigen p24, quantified by immunoenzymatic assay (ELISA, Enzyme-Linked Immunosorbent Assay Perkin Elmer ultrasensitive) in the culture supernatant in the absence or presence of each peptide, was used as parameter of biological activity. In the ex vivo assay the treatment with the peptides 1413, 2733 and 1932 induced, after 5 days of culture, a significant increase in viral replication, with p24 values respectively equal to 191, 261 and 137 picograms/ml, in comparison with 76 picograms/ml found for control cells. Thereafter viral replication tended to decrease and, on day 10, p24 values for the cultured treated with the three peptides were not significantly different from those obtained for the control cells (respectively 67, 98 and 78 pg/ml vs 79 picograms/ml).

The temporary stimulation of viral replication is extremely interesting, as substances able to activate latent viruses have been proposed as antiretroviral therapy adjuvants for their ability to prevent the establishment of persistent reservoirs and allow the final eradication of the virus from the cells.

In the in vitro assay performed by infecting PBMCs from healthy donors with the HIV strain IIIB, the peptides 1413, 2733 and 1932 exhibited a relevant effect of inhibition of viral replication at +8 days of culture, and such inhibitory effect remained unaltered until day 12 in the samples treated with the peptides 1413 and 2733 (FIG. 1).

The results described were confirmed in all experiments conducted by following the two different cell infection and treatment protocols. An analogous behaviour was also observed in the in vitro assays conducted by using the HIV BaL strain; in particular, the peptides 1413 and 2733 and 1923 proved effective in inhibiting viral replication at 12 days, regardless of the protocol applied (FIG. 2).

The assay of endogenous ex vivo replication reported in FIG. 6 highlights the effects of peptides 1932, 2733 and 1413 on viral replication at day 5 of culture. P24 levels on day 10 of culture tend to decrease like in the untreated control. In the figure, there are shown also the results obtained from the treatment of the cells with the incative peptides (values similar to control).

6. Cytotoxicity Test I

Treated cells were subjected to MTT assay at +24 and +48 hours from peptide inoculation. Without removing the medium (IMDM-Iscove's Modified Dulbecco Medium) an amount of 20 μl of MTT solution (3-(4,5-dimethylthiazol-2-yl)-2,5-diphenyltetrazolium bromide) (5 mg/ml) in PBS was introduced in each well for assessment of cell viability after contact with the peptide samples. The test with MTT is specific for hydrophilic substances and is based on the intracellular reduction of tetrazolium salts into formazan crystals by the mitochondrial enzyme succinate dehydrogenase (SDH). After a 3-hour incubation at 38° C., formazan crystals were solubilized with HCl solution in isopropanol (0.04 N, 200 μl) and the stained solution was measured by a microplate reader (Packard Spectracount™, Packard BioScience Company, Meriden U.S.A.) with a 570 nm filter. Viable cells, unlike intoxicated ones, reduce tetrazolium salts; hence, the reaction leading to the blue-colored precipitate will be possible only at the level of metabolically efficient cells, therefore still showing mitochondrial activity. Formazan salt accumulates in cells as it is not able to cross the plasma membrane. In the MTT citotoxicity test at +24 and +48 hours, averages of the values obtained by photometer reading of the data related to tested peptides were compared. NRU (Neutral Red Uptake) assay was performed according to the protocol by Borenfreund and Puerner (1985): a (0.4%) aqueous solution of neutral red was added to each well to a concentration of 50 μg/mL. All was incubated for 3 hours at 38° C., then supernatant was removed. Intracellular Neutral Red was extracted from cells and solubilized with a solution (200 μL) of 1% acetic acid in ethanol 50%. The NRU test is specific for lipophilic substances which are able to modify cell membrane integrity, and is based on the staining of viable cells that, incubated in the presence of Neutral Red, absorb and retain it thanks to cell lysosomes, which then release it following addition of a destaining solution. Only viable cells with perfectly functioning lysosomes are able to capture the dye (assuming a typical red stain) and then release it. When lysosomes break, the cell itself is destroyed, as the hydrolytic enzymes that they contain are able to split all main compounds present in the cell. From a determination of the amount of Neutral Red retained in the cells exposed thereto (in comparison with the control) the relative toxicity of the chemical substances at issue can be assessed.

All tests were performed by measuring the absorbance values in samples without cells (blanks), a sample of cells without peptide (negative control); cells treated with a known toxic substance (positive control), and finally peptide-treated cells. The measurement was performed using a microplate reader (Packard Spectracount™, Packard BioScience Company, Meriden U.S.A.) with a 540-nm filter. The NRU solution was prepared with 0.4% (w/v) Neutral Red in distilled water. Then, the average and the standard deviation referred to each sample was calculated and statistical analysis performed with ANOVA.

Some results of the cytotoxicity tests performed are summed up in FIGS. 3 and 4.

7. Cytotoxicity Test II

Peptide cytotoxicity was evaluated by the Alamar Blue cell viability assay (Biosource International, Inc.); before treatment with the peptides, the PBMCs from healthy subjects were cultivated for 24 h in a 96-well plate at the concentration of 2×10⁶ cells/ml in RPMI1640 additioned with 10% FBS, glutamine and 1% antibiotics, and 20 Ul/ml of rIL-2; then, the peptides were dispensed into the respective wells at increasing concentrations of from 1 to 20 μg/ml and incubated for 20 h at 37° C. under 5% CO₂. Alamar Blue was finally distributed into the wells (10% v/v) and the plate was incubated for 4 h at 37° C. under 5% CO₂. Absorbance was measured with a plate reader (Tecan Sunrise Absorbance Reader) at the dual wavelength of 570/595 nm. Alamar Blue additioned to RPMI1640 complete medium was used as blank. (See FIG. 5).

8. Cytotoxicity test III

By hemolysis experiment on human erythrocytes, it was checked that the peptides at issue possess no cytotoxic effect on the erythrocytes themselves.

The checking was carried out through a spectrophotometry test in which absorbance is read at 405 nm (corresponding to the absorption peak of hemoglobin). Should the peptide(s) have cytotoxic activity towards red cells, the latter would be lysed and would release in the suspension medium hemoglobin that, therefore, would be spectrophotometrically detectable.

To calculate percentage hemolysis the following formula is used:

[(A₄₅₀treated sample−A₄₀₅PBS)/A₄₅₀Tween−A₄₀₅PBS)]×100=%

Therefore, in the graph reported, a low level of cytotoxicity corresponds to a low percentage.

100% hemolysis is obtained by using 0.2% Triton X-100 (positive control). The negative control is instead given by the suspension buffer (PBS).

From the graph reported in FIG. 7, it appears that all three peptides have no hemolytic effect even at concentration well above those useful to induce antiviral effect, and therefore are potentially useful for a parenteral systemic use.

9. Peptide Stability

Current drugs in use for the therapy against HIV infections and AIDS, like e.g. the HAART mixture, are administered in daily doses, whereas the action of the peptides of SEQ ID NO 1, 2, and 3 in in vitro assays highlights a duration of the effect on HIV replication of at least four days.

The effect was verified in two in vitro experimental protocols envisaging:
a treatment of the cells with the virus (adsorption phase), washing, inoculation of the peptide in culture and analysis of viral replication over time;
exposure of the cells to the peptide, adsorption of the cells to the virus, washing, and analysis of viral replication over time.

In both cases, the effect of the peptides on viral replication is identical, demonstrating peptide stability of at least 4 days in the culture medium and indicating that their action should not be bound to the presence of binding sites on cells which inhibit viral entry, but rather by an entry into the cell which at the cytoplasmic and perinuclear level interferes with the viral replication mechanism (as confirmed by investigations with fluorescent peptides under confocal microscope, not reported here).

10. Evaluation of Antiviral Activity

Antiviral activity was evaluated with ex vivo proliferation and in vitro infection assays.

Concerning the ex vivo assays, PBMCs from HIV-1 R5 positive patients, characterized by a high viral load at the plasma level and in the acute infection phase, were cultivated at 37° C. under 5% CO₂, in a 96-well plate at the concentration of 1×10⁶/ml in RPMI 1640 medium supplemented with 10% FBS, glutamine and 1% antibiotics and rIL-2 (20 U/ml). rIL-2 (20 U/ml) was added to the culture every 3-4 days. At t₀ the cells were treated with the peptides at the concentration of 1 and 10 μg/ml for the entire length of the culture. Virus production was evaluated in culture supernatants at days 5 and 10 by p24 ELISA Ultrasensitive assay (Perkin Elmer Life Sciences, Inc. Boston).

In vitro infection assays were conducted by applying two different experimental protocols. PBMCs from three donors were isolated by density-gradient purification (Ficoll), mixed among them in equal amounts to form a pool and cultivated on RPMI1640 complete medium. Prior to infection they were stimulated for 24 h with PHA (5 μg/ml) and thereafter with rIL-2 (20 Ul/ml).

The first protocol consisted in infecting the PBMCs with HIVIIIB (X4) or BaL (R5) (MOI=0.5) for 2 h, washing them with PBS1X and cultivating them in a 96-well plate for 12 days with the addition of 10 μg/ml of peptide and 20 Ul/ml of rIL-2 every 4 days.

The second protocol consisted instead in pre-incubating the cells for 2 h at 37° C. with the peptides, infecting them for 2 h with HIVIIIB or BaL (MOI=0.5), washing with PBS1X and cultivating them in a 96-well plate for 12 days. In this case, every 4 days only rIL-2 (20 Ul/ml) is additioned to the wells. In both protocols, culture supernatants are harvested at days 8 and 12 of infection.

Viral replication was evaluated by measuring the concentration of antigen p24 in culture supernatants with the test HIV p24 ELISA Ultrasensitive detection kit (Perkin Emer, Inc. Boston), following the provider's indications.

In order to identify a putative action mechanism, first of all the tendency of peptide-treated CD4+ T lymphocytes to form syncytia with respect to untreated controls was evaluated. For this purpose, CHO 33T, HeLa ADA and HeLa LAI cell lines, which constitutively express the gp120 of HIV on their surface, were used. The cells were cultivated for 24 h in a 6-well plate (25×10⁴ cell/well) in high glucose D-MEM medium supplemented with 10% FBS, glutamine, antibiotics and 1% sodium piruvate. The next day, CD4+ cells from three donors were isolated by positive selection (Miltenyi Biotech Inc.), mixed to set up a pool and co-cultivated with the CHO 33T, HeLa ADA or HeLa LAI cells in a RPMI 1640 complete medium to the concentration of 10⁶ cell/well in the presence of the peptides (10 μg/ml). Syncitia formation was observed after 18 h of incubation at 37° C. under 5% CO₂.

A set of experiments is currently under way, aimed at identifying the cell localization of the peptides at issue. These tests make use of a confocal microscope equipped with an incubator, allowing to carry out a dynamic assessment of the migration of the peptide inside the cell. Preliminary assays were performed under fluorescence, using the HeLa cell line incubated with the fluorescinated peptide 1932. For this purpose, the HeLa cells were seeded on 8-well chamber slides (A=0.8 cm²/well) at a concentration of 25×10³ cells/well. The next day, the medium was sucked up from the wells, replaced with fresh medium without serum, containing the peptide at a concentration of 10 μg/ml. The chamber slides were incubated at 37° C. for 4 h under 5% CO2, and washed 3 times with PBS1X for 5 min. Finally, the slide was mounted with Fluorescent Mounting Medium (Dako) and observed under microscope.

### REFERENCES

Bonetto V., Andersson M., Bergman T., Sillard R., Morberg A., Mutt V. et al. Spleen antibacterial peptides: high levels of PR39 and presence of two form of NK-lysin. Cell. Mol. Life. Sci 1999, 56: 174-178.
Helmerhorst E J, Sun X, Salih E, Oppenheim F G. Identification of Lys-Pro-Gln as a novel cleavage site specificity of saliva-associated proteases. J. Biol. Chem. 2008, 283: 19957-66.
Jeffreys Richard, Journal of the International AIDS society—Workshop report: “Towards a cure: HIV reservoirs and strategies to control them” 2010, Vol 13 (Suppl. 3) I1 http://www.jiasociety.org/content/13/S3/I1
Messana I, Cabras T, Inzitari R, Lupi A, Zuppi C, Olmi C, Fadda M B, Cordaro M, Giardina B, Castagnola M. Characterization of the human salivary basic proline-rich protein complex by a proteomic approach. J Proteome Res. 2004, 3:792-800.
Messana I, Cabras T, Pisano E, Sanna M T, Olianas A, Manconi B, Pellegrini M, Paludetti G, Scarano E, Fiorita A, Agostino S, Contucci A M, Calò L, Picciotti P M, Manni A, Bennick A, Vitali A, Fanali C, Inzitari R, Castagnola. Trafficking and post secretory events responsible for the formation of secreted human salivary peptides: a proteomics approach. Mol Cell Proteomics. 2008, 7:911-926.
O'Sullivan J M, Cannon R D, Sullivan P A, Jenkinson H F. Identification of salivary basic proline-rich proteins as receptors for Candida albicans adhesion. Microbiology. 1997, 143:341-348
Robinovitch M R, Ashley R L, Iversen J M, Vigoren E M, Oppenheim F G, Lamkin M. Parotid salivary basic proline-rich proteins inhibit HIV-I infectivity Oral Dis. 2001, 7: 86-93.
Nitin K Saksena, Bin Wang, Li Zhou, Maly Soedjono, Yung Shwen Ho, Viviane Conceicao “HIV reservoirs in vivo and new strategies for possible eradication of HIV from the reservoir sites” HIV/AIDS—Research and Palliative Care 2010, pp 103-122.
Shi J., Ross C. R., Chengappa M. M., and Blecha F., Identification of a proline-arginine-rich antibacterial peptide from neutrophils that is analogous to PR39, and antibacterial peptide from the small intestine. J. Leukoc. Biol. 1994, 56: 807-811 ( ).
Stangler T, Tran T, Hoffmann S, Schmidt H, Jonas E, Willbold D. Competitive displacement of full-length HIV-1 Nef from the Hck SH3 domain by a high-affinity artificial peptide Biol. Chem. 2007, 388:611-5.
Vitorino R, Barros A, Caseiro A, Domingues P, Duarte J, Amado F. Towards defining the whole salivary peptidome. Proteomics, Clin. Applic. 2009, 3: 528-540.
U.S. Pat. No. 5,981,720
White M R, Helmerhorst E J,Ligtenberg A, Karpel M, Tecle T, Siqueira W L, Oppenheim F G, Hartshorn K L. Oral Microb. Multiple components contribute to ability of saliva to inhibit influenza viruses. Immun. 2009, 24: 18-24
Yang B, Gao L, Li L, Lu Z, Fan X, Patel C A, Pomerantz R J, DuBois G C, Zhang H. Potent suppression of viral infectivity by the peptides that inhibit multimerization of human immune deficiency virus type 1 (HIV-1) Vif proteins. J Biol Chem. 2003, 278:6596-6602.
Zerman A., Skerlavaj B., Gennaro R., Romeo D., Inactivation of Herpes simplex virus by protein components of bovine neutrophil granules. Antiviral Res 1987, 7: 341-352.

1. A peptide of SEQ ID NO 1 with antiviral and antimycotic activity.

2. A method of using the peptide according to claim 1, comprising administering said peptide.

3. The method according to claim 2 for use in treatment and/or prevention of HIV infection and/or for eradication of HIV virus from viral reservoirs in individuals suffering from HIV and/or in therapy for AIDS.

4. The peptide according to claim 2 for use in a therapeutic method for eradication of HIV reservoirs and/or for treatment and/or prevention of HIV infections and/or in therapy for AIDS, comprising administering to a patient in need thereof a pharmacologically effective dose of said peptide.

5. A nucleotide sequence coding for the peptide of SEQ ID NO 1.

6. A pharmaceutical composition comprising an antiviral peptide of SEQ ID NO 1 as active principle and a pharmaceutically acceptable carrier.

7. The pharmaceutical composition according to claim 6, further comprising one or more of excipient, preservative, thickener, adjuvant, vehicle, diluent, solvent, aiding agent for dispersion or suspension, surface active agent, isotonic agent, emulsifying agent, solid binder, or lubricant.

8. The pharmaceutical composition according to claim 6 for oral, parenteral, intravenous, aerosol, rectal, transdermic, subcutaneous, intracisternal, intramuscular, intravaginal, intraperitoneal, topical, perilingual, or intranasal use.

9. The pharmaceutical composition according to claim 6, wherein the peptide of SEQ ID NO 1 represents from about 0.01 to about 50% by weight of said composition.

10. The pharmaceutical composition according to claim 6, further comprising one or more of the active principles selected from the group consisting of: peptide of SEQ ID NO 2, peptide of SEQ ID NO 3, nucleoside inhibitors; non-nucleoside inhibitors; reverse transcriptase inhibitors; integrase inhibitors; viral protease inhibitors; antimycotics; antibacterials; and virus-host cell fusion process inhibitors.

11. The pharmaceutical composition according to claim 10, wherein said peptide of SEQ ID NO 1 represents at least 51% of the active principles.

12. The pharmaceutical composition according to claim 6 for use in treatment and/or prevention of HIV infection and/or for eradication of HIV virus from viral reservoirs in individuals suffering from HIV and/or in therapy for AIDS.

13. The pharmaceutical composition according to claim 12 for use in a therapeutic method for eradication of HIV reservoirs and/or for treatment and/or prevention of HIV infections and/or in therapy for AIDS, comprising administering to a patient in need thereof a pharmacologically effective dose of said composition.

14. A pharmaceutical kit for concomitant or sequential administration, comprising one or more aliquots of the antiviral peptide of SEQ ID NO 1 as active principle in a pharmaceutically acceptable carrier and one or more aliquots of one or more of the active principles selected from the group consisting of: peptide of SEQ ID NO 2, peptide of SEQ ID NO 3, nucleoside inhibitors; non-nucleoside inhibitors; reverse transcriptase inhibitors; integrase inhibitors; viral protease inhibitors; antimycotics; antibacterials; and virus-host cell fusion process inhibitors.

15. The pharmaceutical kit according to claim 14, wherein each aliquot further comprises one or more of excipient, preservative, thickener, adjuvant, vehicle, diluent, solvent, aiding agent for dispersion or suspension, surface active agent, isotonic agent, emulsifying agent, solid binder, or lubricant.

16. The pharmaceutical kit according to claim 14 for oral, parenteral, intravenous, aerosol, rectal, transdermic, subcutaneous, intracisternal, intramuscular, intravaginal, intraperitoneal, topical, perilingual, or intranasal use.

17. The pharmaceutical kit according to claim 14 for use in treatment and/or prevention of HIV infection and/or for eradication of HIV virus from viral reservoirs of individuals suffering from HIV and/or in therapy for AIDS.

18. The pharmaceutical kit according to claim 14 for use in a therapeutic method for eradication of HIV reservoirs and/or for treatment and/or prevention of HIV infections and/or in therapy for AIDS, comprising administering concomitantly or sequentially to a patient in need thereof an aliquot comprising the antiviral peptide of SEQ ID NO 1 in a pharmaceutically acceptable carrier and one or more aliquots comprising one or more of the active principles selected from the group consisting of: peptide of SEQ ID NO 2, peptide of SEQ ID NO 3, nucleoside inhibitors; non-nucleoside inhibitors; reverse transcriptase inhibitors; integrase inhibitors; viral protease inhibitors; antimycotics; antibacterials; and virus-host cell fusion process inhibitors.


SEQUENCE LISTING (14 sequences)

SEQ ID NO: 1 - protein, 20 aa, Homo sapiens
GPPPQGGNKPQGPPPPGKPQ

SEQ ID NO: 2 - protein, 28 aa, Sus sp.
DKPKKKPPPPAGPPPPPPPPPGPPPPGP

SEQ ID NO: 3 - protein, 15 aa, Artificial Sequence
  proline rich peptide non natural variant of a 1511 Da peptide from Sus sp.
RPPPGPPPPGPPPGP

SEQ ID NO: 4 - protein, 4 aa, Artificial Sequence
  putative domain for inhibition of HIV glicosylase in US 5,981,720
GGNK

SEQ ID NO: 5 - protein, 17 aa, Homo sapiens
GPPPQGGNKPQGPPPPG

SEQ ID NO: 6 - protein, 11 aa, Homo sapiens
GPPPQGGNKPQ

SEQ ID NO: 7 - protein, 16 aa, Homo sapiens
GPPPQGGNKPQGPPPG

SEQ ID NO: 8 - protein, 8 aa, Homo sapiens
GPPPQGGN

SEQ ID NO: 9 - protein, 9 aa, Homo sapiens
GPPPPGKPQ

SEQ ID NO: 10 - protein, 21 aa, Sus sp.
APPGARPPPGPPPPGPPPPGP

SEQ ID NO: 11 - protein, 7 aa, Sus sp.
RSPFFDL

SEQ ID NO: 12 - protein, 22 aa, Sus sp
APPGARPPPPPPPPADQPQQGP

SEQ ID NO: 13 - protein, 21 aa, Sus sp.
APPGARPLPGPPPPGPPPPGP

SEQ ID NO: 14 - protein, 4 aa, Artificial Sequence
  putative consensus motif
PXXP